(19) United States
(12) Plant Patent Application Publication
(10) Pub. No.: US 2016/0316597 P1
(43) Pub. Date: Oct. 27, 2016

(54) Sweet cherry 'IFG Cher-one'

(50) ;

(71) Applicant: David Cain, Bakersfield, CA (US)

(72) Inventor: David Cain, Bakersfield, CA (US)

(21) Appl. No.: 14/545,349

(22) Filed: Apr. 27, 2015

(51) Int. Cl. A01H 5/00

(52) U.S. Cl. PLT/181

(57) ABSTRACT

This invention is a new and distinct sweet cherry variety denominated ‘IFG Cher-one’. The new sweet cherry is characterized by producing very large dark red kidney shaped fruits having broad shoulders and flat to round base. Fruits ripen early about three days ahead of the Brooks variety.

### LATIN NAME OF THE GENUS AND SPECIES CLAIMED

Prunus avium

### VARIETY DENOMINATION

‘IFG Cher-one’

### BACKGROUND OF THE INVENTION

The new and distinct sweet cherry described and claimed herein originated from open pollinated seeds collected in May 2001, from a pollenizer tree designated as 10-1 in a commercial orchard of the Brooks variety (U.S. Plant Pat. No. 6,676).growing near Delano, Kern County, Calif. The seeds were stratified, germinated and the resulting seedlings were planted in the field at the International Fruit Genetics research facility located near Delano, Kern County, Calif. in April 2002. The present variety of sweet cherry was selected as a single plant in May 2005 and was first asexually propagated in January 2006 by grafting onto a tree of Belle de Planchoury, itself being grafted on Prunus mahalab rootstock. It was subsequently propagated directly onto Prunus mahalab rootstock by chip budding in April 2007. These propagules were found to reproduce true-to-type by asexual propagation. All propagation was done at the International Fruit Genetics research facility located near Delano, Kern County Calif.

### BRIEF SUMMARY OF THE INVENTION

Sweet cherries have traditionally been grown in climates with long cold winters and cool to moderately warm summers. Such climates provide enough cold winter temperatures to allow normal growth to resume in the spring and summer temperatures that are low enough not to induce production of unmarketable double or spurred fruit, but it limits the seasonality that cherries are available. The International Fruit Genetics sweet cherry breeding program focuses on developing types of cherries that will grow in regions with low winter chilling and high summer temperatures so that the fruit will ripen before fruit in traditional growing regions.

The new sweet cherry ‘IFG Cher-one’ is characterized by producing very large dark red kidney shaped fruits having broad shoulders and flat to round base. Fruits ripen early about three days ahead of the Brooks variety. The ‘IFG Cher-one’ has somewhat soft acidic flesh. Firmness can be improved by application of gibberellic acid. The tree has a medium high chilling requirement similar to or slightly lower than the Brooks variety. It produces a similar number of doubled and spurred fruits as the Brooks variety in high summer temperature regions such as the Southern San Joaquin Valley of California. Fruits of ‘IFG Cher-one’ have medium long, thick stems that remain attached and stay green during storage and shipping.

In comparison to the Brooks variety, which is a major variety grown in warm regions, the present variety ripens about three days earlier, is larger and has more desirable darker red skin and flesh. In comparison to its female parent, the present variety has a lower chilling requirement and exhibits less undesirable symptoms of lack of chill including more consistently setting crops, exhibiting less undesirable variable ripening and producing fewer unmarketable doubled or spurred fruits.

In comparison to the Tulare variety (U.S. Plant Pat. No. 6,407), the ‘IFG Cher-one’ produces larger fruit that ripen approximately six to eight days before the Tulare variety. The fruit shape of ‘IFG Cher-one’ has a more desirable round to kidney shape with broad shoulders and flat stylar end compared to the more pointed, narrower shoulder shape of the Tulare variety. The ‘IFG Cher-one’ has a higher chill requirement and produces more doubled fruits than the Tulare variety.

### BRIEF DESCRIPTION OF THE FIGURE

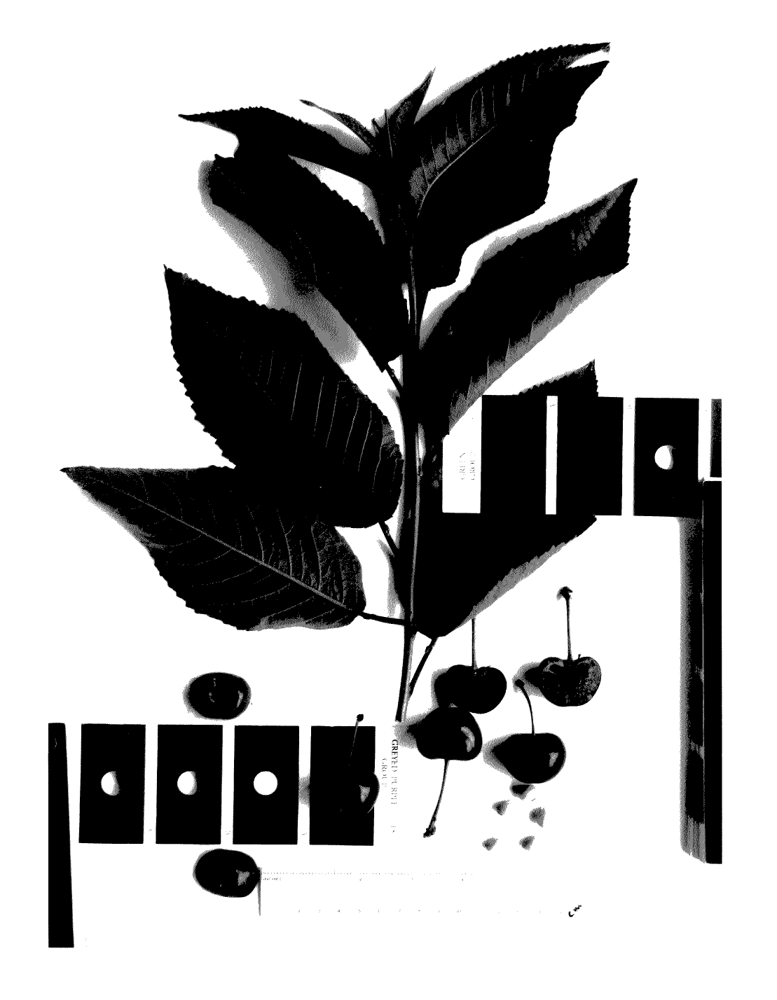

The accompanying photographic illustration in FIG. 1 illustrates in full color ‘IFG Cher-one’. The photograph was taken outdoors with indirect lighting. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Board for Plant Genetic Resources (IBPGR) ‘Cherry Descriptor List’ (Prunus spp.) (1985) which was developed with full support from the Commission of the European Communities (CEC) Programme Committee for Plant Disease Resistance Breeding and the Use of Genebanks.

The descriptive matter which follows pertains to an ‘IFG Cher-one’ plant growing on its own roots grown in the vicinity of Delano, Kern County, Calif. during 2013 and 2014, and is believed to apply to plants of the variety grown under similar conditions of soil and climate elsewhere:
Tree:
   General.—Age — 11 yrs. old Height — About 2.3 M Width — About 3.2 M Vigor — Vigorous Density of foliage — Dense Form — Spreading Productivity — Productive Root stock — Own root Resistance to — Insects: Average typical of Prunus avium species. Diseases: Average typical of Prunus avium species. Chilling requirement — Medium, similar to ‘Brooks’ Graft compatibility — Good; produces compatible graft unions with Mazzard seedlings (non-patented) and Mahaleb seedlings (non-patented).
   Trunk.—Trunk diameter of 12 year old trees, 30 cm above the soil line — About 19.9 cm Lenticel size — Large Lenticel dimensions — Length: About 1.3 cm. Width: About 0.3 cm Lenticel shape — Elliptical shape oriented horizontally Lenticel color — Can be any of the following colors: 156A and C Trunk surface texture — Rough Outer bark color — Can be any of the following colors: 187B and 183B and 198C.
Branches:
   1 year old wood.—Vertical top growth length — Long; About 146.4 cm Horizontal growth length — Medium; About 47.7 cm Diameter — Vertical growth: About 1.6 cm. Horizontal growth; About 0.9 cm Internode length — Medium: About 3 cm Number of lenticels — Few Lenticel size — Small Lenticel dimensions — Length: About 1 mm. Width: About 2 mm Lenticel shape — Round Bark color — Can be any of the following colors: 175A and 166B.
   2 year old wood.—Length — About 51.0 cm Diameter — About 1.1 cm Internode length — About 3.2 cm Number of lenticels — Medium dense Lenticel dimensions — Length: About 5 mm. Width: About 1 mm Lenticel shape — Elliptical shape oriented horizontally Bark color — Can be any of the following colors: 177A and 165A and 164A.
Buds:
   Vegetative buds.—Shape — Elongated Vegetative bud dimensions — Length: About 7 mm. Width: About 3 mm Vegetative bud burst — Feb. 25, 2014.
   Flower buds.—Flower bud dimensions — Length: About 10.6 mm. Width: About 7.1 mm Shape — Oval Placement — At bud positions mostly 1 to 6 on 1 year wood Average number of flower buds on first year wood — About 3.4 Number of flower buds per spur on second year wood — 2 to 7. Average: About 4.6 Color — Can be any of the following colors: 144A and B Flower bud burst — Feb. 25, 2014.
Leaves:
   Mature leaves.—Leaf dimensions — Length; About 16.9 cm. Width; About 8.3 cm Leaf shape — Elliptic: Symmetric on both sides of central axis Shape of tip — Acute; broadly Shape of base — Oblique Margin — Serrated; regular Surface texture — Smooth Leaf profile — Involute.
   Upper surface.—Upper surface pubescence — None Upper leaf surface color — 147A.
   Lower surface.—Lower surface pubescence — All over; medium dense Lower leaf surface color — 147B.
   Petiole.—Petiole dimensions — Length: About 3.0 cm. Width: About 2.8 cm Upper surface of petiole color — 177A Lower surface of petiole color — 166A Petiole groove — Wide Petiole pubescence — Only on lower surface; sparse to medium Venation — Arcuate Vein color — 145C.
   Glands.—Number of glands — 1 to 2 Gland dimensions — Length; About 3.3 mm. Width; About 2 1 mm Gland shape — Reniform Gland location — On petiole Gland color — Can be any of the following colors; N34 and 153A Leaf stipule — Not present.
Flowers: Blooming period — Mid-season Blooming dates — First bloom: 3-5-13. Full bloom 3-15-13 Number of flowers per cluster — 2 to 4. Average: About 2.5 Corolla diameter — About 3.3 cm Petal number — 5 Petal length — About 1.8 cm Petal width — About 1.6 cm Margin waviness — Weak Division of upper margin — Notched Color of petal upper surface — 155D Color of petal lower surface — 155D Peduncle — Length: About 1.1 cm. Width: About 0.12 cm Peduncle Color — 144A Number of sepals — 5 Sepal length — About 0.6 cm Sepal width — About 0.5 cm Sepal shape — Triangular Sepal color — Upper surface: 144C. Lower surface 60C Filament — Length: About 0.6 to 1.5 cm. Width: About 0.03 cm Filament color — 155D Anther color — 17C Pollen color — Can be any of the following colors: 162A and 163A Pollen production — Medium Flower compatibility group — S6S9
Fruit:
   General.—Ripening period — Early midseason about 3 days before ‘Brooks’: Approximately: 5-3-13 Use — Fresh market Shipping quality — Average % Titratable acidity — About 0.70% Refractometer test — Soluble solids; Brix — About 14.4 Firmtech II (g/mm) — About 311 Flavor — Average, slightly acidic Juice color — Dark red/purple Juice amount — Intermediate Eating quality — Average.
   Stem.—Stem — Length: About 2.8 cm. Width: About 0.17 cm Stem color — 144A Stem cavity — Wide Stem retention during storage — Very good Stem storage quality — Good.
   Berry.—Uniformity of size — Uniform Shape — kidney Fruit weight — About 10.1 gm Apical diameter — About 26.7 mm Diameter transversely across suture — About 21 7 mm Diameter at right angle to suture plane — About 26.7 mm Suture — None Percent of excessively deep or split sutures — About 0% Doubles — About 16%, similar to ‘Brooks’.
   Skin.—Thickness — Medium Texture — Medium Skin Color — Can be any of the following colors; 59A and B and C Tendency to tip crack — Not susceptible Tendency to stem cavity crack — Not susceptible.
   Flesh.—Texture — Intermediate Color — Can be any of the following colors: 187A and B.
   Stone.—Shape — Oblong Stone dimensions — Length: About 12 mm. Width: About 8 mm Type — Clingstone Surface texture — Smooth Stone color when dry — 159B Tendency to split — None Shape — Round to slightly ovate Base — Flat Apex — Rounded Ventral edge — Narrow suture subtended by 2 somewhat prominent ridges converging at base and apex Dorsal edge — Smooth, narrow ridge from base to apex.

What is claimed:

1. A new and distinct variety of sweet cherry as herein illustrated and described.
